(19) United States
(12) Patent Application Publication
(10) Pub. No.: US 2004/0101559 A1
(43) Pub. Date: May 27, 2004

(54) Novel pharmaceutical formulations

(76) Inventor: David Wong, Milpitas, CA (US)

Correspondence Address: David W Barman, 1101 NE 176 Terrace, North Miami Beach, FL, 33162, US

(21) Appl. No.: 10/303,467

(22) Filed: Nov. 25, 2002

(51) Int. Cl. A61K 9/14

(52) U.S. Cl. 424/486

(57) ABSTRACT

This present invention relates to polymer-clay composites for pharmaceutical dosage forms, dosage forms produced from the composites; and processes for preparing the composites, and dosage forms.

### FIELD OF THE INVENTION

This invention relates generally to polymer-clay composites comprising a mixture of clay materials and pharmacological active substance. This invention further relates to polymer-clay composites, and dosage forms produced from the composites; and processes for preparing the composites, and dosage forms.

### BACKGROUND OF THE INVENTION

Polymer clay techniques have been widely used in art and craftsmanship for several years. As its name implies, polymer clay is a pliable and blendable polymer compound. It is not a true clay—clay is fine silicate particles suspended in water, whereas polymer clay is fine polyvinyl chloride (PVC) suspended in plasticizer—but it can be used much like clay.

Polymer clay is versatile. It can be blended with dozens of colors like paints to make the desired colors. Since the color is inherent in the particles, the artists or crafters can also work two or more colors together without blending them, for special effects such as cane working and marbling. The clay's pliability and ductility allow artists or crafters use techniques from glasswork, textile arts, and sculpture. And polymer clay doesn't dry out, so they can sculpt and form it without worrying about a time limit.

Artists or crafters can cover anything (as long as it won't melt or burn at the low firing temperatures) with a veneer of polymer clay: wooden boxes, picture frames, mirrors, and tableware. One popular application is jewelry: polymer clay can be used to make beads, pendants, bracelets, and neckpieces. Small sculptures and buttons are other possibilities. Clay artists have developed techniques to give polymer clay the appearance of granite, jade, amber, coral, turquoise, and ivory, and its flexibility means they can make pieces in shapes and sizes that wouldn't be possible using actual stone.

One important step in the process is “firing”. It is the process that fuses the particles into a solid—requires only low temperatures. The colors and size do not change during firing. When fired, the clay gets hard enough to make durable objects, and can be finished in various ways to obtain textures from glassy to stone-like.

There are many examples in the patent literature of polymer/clay composites. For example, U.S. Pat. No. 4,739,007 discloses the preparation of Nylon-6/clay nanocomposites from caprolactam and alkyl ammonium-treated montmorillonite. U.S. Pat. No. 4,889,885 describes the polymerization of various vinyl monomers such as methyl methacrylate and isoprene in the presence of sodium montmorillonite. Some patents describe the blending of up to 60 weight percent of intercalated clay materials with a wide range of polymers including polyamides, polyesters, polyurethanes, polycarbonates, polyolefins, vinyl polymers, thermosetting resins and the like. Such high loadings with modified clays are impractical and useless with most polymers because the melt viscosity of the blends increases so much that they cannot be molded. WO 93/04117 discloses a wide range of polymers melt blended with up to 60 weight percent of dispersed platelet particles. WO 93/04118 discloses nanocomposite materials of a melt processable polymer and up to 60 weight percent of a clay that is intercalated with organic onium salts. The use of a mixture of swellable layered clays or a clay mixture intercalated with an onium ion is not contemplated nor disclosed. U.S. Pat. No. 5,552,469 describes the preparation of intercalates derived from certain clays and water soluble polymers such as polyvinyl pyrrolidone, polyvinyl alcohol, and polyacrylic acid. The use of clay mixtures or mixtures intercalated with onium ions is specifically excluded. Polymer clay technology is simple and easy to scale-up to production batches, moreover, there is no application of polymer clay into drug delivery systems.

In pharmaceutical industry, particles composing of pharmacological active substance (inside the matrix) are mainly made by spray granulation, high-shear granulation, slugging (or roller-compaction) and pelletization. In spray granulation, the powder to be granulated is suspended in the heated air of a fluid bed, and a liquid or molten binder sprayed from nozzles positioned above while the high-shear granulation process combines the active powder with a binder solution or a molten binder using a high-speed mixing blade and chopper. Pellets are usually prepared by wet granulation in a rotary processor (fluidized bed rotor granulator) or high-shear mixer and then by extrusion/spheronization process. Pellets can also be produced by compression.

There is much interest in applying polymer-clay technology into drug delivery systems because of its simple process. Besides, this process can be suitable for water-liable drugs because water or solvent is not necessarily needed in the process, or just a limit amount of solvent is needed to assist the solvency. On the other hand, plasticizer is always needed in the process. Plasticizer is a material, such as dioctyl phthalate, added to some polymers to increase their flexibility. For example, uPVC, unplasticized PVC, is rigid enough to be used for window frames but would be useless for making the soles of shoes since it is not flexible enough. Plasticizers are chemically and thermally stable and therefore do not undergo reaction during processing. After the addition of plasticizers to the drug/polymer blend, the “dough” will be press

Therefore, as shown above, a need exists for application this technology into pharmaceutical area especially drug delivery systems. This invention provides a novel polymer clay composite comprising a mixture of clay materials and pharmacological active substance. This invention also provides application of this technology on drug delivery systems.

### SUMMARY OF THE INVENTION

It has been discovered that a polymer-clay composite is useful in craftsmanship for the manufacturing of wooden boxes, picture frames, mirrors, tableware, small sculptures and buttons. It is also used to produce jewelry articles such as beads, pendants, bracelets, and neckpieces. With some particular treatments, polymer-clay composites can also be used to make items with similar appearance of granite, jade, amber, coral, turquoise, and ivory.

This invention further applies polymer-clay composites to deliver pharmaceutical active substances. This invention also seeks to provide a simple and cost-effective method for producing pharmaceutical particles containing one or more pharmacological active substances. The polymer-clay composite composition and process of this invention are especially suited for use in applications for delivery of pharmacological active substance.

In accordance with the purpose(s) of this invention, as embodied and broadly described herein, this invention, in one embodiment, relates to a polymer-clay composite comprising (i) a polymer and (ii) one or more pharmacological active substance.

In another embodiment, this invention relates to a process for preparing a polymer-clay composite comprising (i) preparing a mixture of at least one polymer, one pharmacological active substance and optionally a plasticizer, and (ii) incorporating the mixture with a matrix polymer by conditioning the matrix polymer with or without a plasticizer.

Additional advantages of the invention will be set forth in part in the detailed description, which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory of preferred embodiments of the invention, and are not restrictive of the invention, as claimed.

### DETAILED DESCRIPTION OF THE INVENTION

The present invention may be understood more readily by reference to the following detailed description of the invention and the examples provided therein. It is to be understood that this invention is not limited to the specific processes and conditions described, as specific processes and/or process conditions for processing polymer articles as such may, of course, vary. It is also understood that the terminology used herein is for the purpose of describing particular embodiments only and is not intended to be limiting.

It must also be noted that, as used in the specification and the appended claims, the singular forms “a,” “an” and “the” included plural references unless the context clearly dictates otherwise.

Ranges may be expressed herein as from “about” or “approximately” one particular value and/or to “about” or “approximately” another particular value. When such a range is expressed, another embodiment includes from the one particular and/or to the other particular value. Similarly, when values are expressed as approximations, by use of the antecedent “about,” it will be understood that the particular value forms another embodiment.

### DEFINITIONS

Whenever used in this specification or claims, the terms set forth shall have the following meanings:

“Polymers” shall mean any large molecules formed by the union of at least five identical monomers; it may be natural, such as acacia, agar, heparin sodium, pectin, sodium alginate, guar gum, tragacanth, xanthan gum, starch, casein, gelatin, protamine sulfate or synthetic, such as cellulose derivatives, vinyl derivatives carbomer, and polyethylene oxides; polymers usually contain many more than five monomers.

“Plasticizers” or “conditioners” shall mean any chemicals are used in order to achieve polymer softness and flexibility. Usually, they are nonionic. Commonly used plasticizers for film coating include oils, hydrogenated oils, ethyl phthalate, butyl phthalate, glycerides, waxes, water, polyethylene glycols, sorbitan esters, and citrates.

“Polymer-clay dough” or “dough” shall mean individual or aggregate mass or lump, after or during the conditioning process.

“Conditioning process” shall mean the process softens the polymer-clay by applying pressure to the polymer-clay.

“Polymer-clay composite(s)” shall mean a mixture of polymer and plasticizer after firing or solidifying the polymer-clay dough. In this invention, it contains pharmacological active substance.

“Firing” shall mean the process solidifying the polymer-clay dough by heat.

### DESCRIPTION OF THE EMBODIMENTS

The present invention relates to polymer-clay composite compositions comprising at least one polymer, at least one plasticizer and pharmacological active substance, which are combined to form a mixture, a process for preparing a polymer-clay composite, and to product made with said mixture which may be molded articles, films, fiber, etc. or milled fine particles prepared from the polymer-clay composites of this invention. The process of this invention may be used to prepare a wide variety of pharmaceutical dosage forms such as beads, pellets, tablets, micro-particles and suspensions.

Without being bound by any particular theory, it is believed that the drug release rate from the polymer-clay composites depends upon the physical and chemical characteristics of the polymer, plasticizer and drug used in that particular composition.

To improve the dissolution of a water-insoluble drug, it is preferable that the polymer and plasticizer to be water-soluble. It is optional to incorporate surfactants into the composite. Milling the composite into fine particles for improving dissolution is most preferred.

Significant levels of incomplete conditioning (i.e., applying pressure to the polymer-clay to form the dough) not only lead to poor particle interaction, but also can lead to deleterious effects on other properties of the composite such as strength, toughness and process-ability.

In one embodiment, this invention relates to a polymer-clay composite comprising a polymer and up to about 25 weight percent of a plasticizer and 10 weight percent of pharmacological active substance, which may be conditioned by press the dough with fingers. The dough is heated at 50° C. and solidified within an hour.

In another embodiment, a process for manufacturing the polymer-clay composite of this invention may comprise: (1) preparing the polymer-clay composite and (2) incorporating the layered clay material mixture with a pharmacological active substance by pressuring the polymer-clay dough with a rolling pin. To ensure the even thickness of the sheet, a dowel is placed to either side of the poly-clay dough to rest the roller on the dowel. This flattens the clay sheet to the desired thickness.

The composite composition of the present invention comprises one or more pharmaceutically acceptable polymer(s) mixed with one or more suitable plasticizer(s) and one or more pharmacological active substance(s). The selection of polymers depends on the objective of drug delivery. For instances, enteric polymer(s) should be used for delivery of pharmacological active substance to the lower GI tract, and higher molecular weight water-soluble polymer(s) should be used for slow drug release profile.

Polymers that may be used in one embodiment of the invention include acacia, agar, heparin sodium, pectin, sodium alginate, guar gum, locust seed gum, tragacanth, xanthan gum, starch, casein, gelatin, protamine sulfate or synthetic, such as cellulose derivatives, vinyl derivatives carbomer, and polyethylene oxides. Suitable polymers may contain, in one embodiment, many more than five monomers.

Plasticizers suitable for one embodiment of the present invention may include alcohols, oils, hydrogenated oils, ethyl phthalate, butyl phthalate, glycerides, waxes, water, polyethylene glycols, sorbitan esters, and citrates. The selection of plasticizers depends on the polymer(s) used for the polymer-clay composites. In one preferred embodiment only one plasticizer is used in the composite.

Other non-clay materials such as surfactants and fillers may also be used to modify the drug dissolution profiles or/and the mechanical strengths of the finished products e.g. beads.

This invention also relates to nano, micro, and larger particles prepared from the composite of this invention, including, but not limited to, pellets, micro or nano particles, or even molded articles such as suppositories. The pellets or beads can be encapsulated into capsules or compressed into tablets for oral drug delivery. If the composite is milled to 50-200 micron, the milled particles will have a good flow. And due to their good binding properties, certain milled particles are extremely suitable for compression or tableting. Development and production of such tablets uses generally available techniques and equipment.

The finished products, i.e. tablets or capsules, may also contain different types of polymer-clay particles for some unique drug dissolution profiles. For instances, immediate release polymer-clay particles can be mixed with sustained release polymer-clay particles to form a sustained release profile with an initial burst effect or immediate release polymer-clay particles can be mixed with delay release composite to form stepwise drug release profiles.

In another embodiment of this invention, the polymer-clay particles are coated with enteric or sustained release polymer for some specific drug release profiles. Development and production of such coated particles uses generally available techniques and equipment.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A polymer-clay composite comprising:
(a) at least one polymer;
(b) at least one plasticizer; and
(c) one or more pharmaceutically active ingredients.

2. The polymer-clay composite of claim 1, wherein the mixture comprises any pharmaceutically acceptable polymers.

3. The polymer-clay composite of claim 1, wherein the pharmaceutically acceptable polymers can be water-soluble or water-insoluble.

4. The polymer-clay composite of claim 1, wherein the plasticizer(s) is/are pharmaceutically acceptable.

5. A process for preparing a polymer-clay composite comprising:
(i) preparing a mixture of at least one polymer with at least one plasticizer and
(ii) incorporating the mixture with one or more pharmaceutically active ingredients.

6. The process of claim 5, wherein said plasticizer can be replaced by a solvent.

7. The process of claim 6, wherein the solvent is water, an alcohol, a chlorinated solvent, a ketone, an ester, an ether or mixtures thereof.

8. A polymer-clay composite made by the process of claim 5.

9. A polymer-clay composite made by pressuring or conditioning the polymer blend made by the process of claim 5.

10. The polymer-clay composite of claim 1, wherein the pharmaceutically acceptable polymers can be softened by the plasticizer or solvent added.

11. A method of making a polymer-clay composite comprising:
(a) mixing at least one polymer, plasticizer (or solvent) together with at least one pharmacological active substance,
(b) pressuring the blend into a dough,
(c) and firing the dough to form a polymer-clay solid which is suitable to be incorporated into a pharmaceutical dosage form.

12. The method of claim 11 wherein said polymer-clay solid is sized for a pharmaceutical dosage form.

13. The method of claim 12 wherein said sizing of said polymer-clay solid comprises milling or extruding said composite.

14. The method of claim 13 wherein said polymer-clay solid sized for a dosage form is further incorporated into a dosage form.

15. The method of claim 14 wherein said dosage form is a capsule, tablet or suspension.

16. The dosage form of claim 13 wherein said composite is incorporated into a suspension.
